(19) United States
(12) Patent Application Publication
(10) Pub. No.: US 2021/0379162 A1
(43) Pub. Date: Dec. 9, 2021

(54) TRANSDERMAL SYSTEM FOR THE DELIVERY OF ABALOPARATIDE AND METHOD OF USE FOR TREATING OSTEOPOROSIS

(71) Applicants: Radius Health, Inc., Boston, MA (US); Kindeva Drug Delivery L.P., St. Paul, MN (US)

(72) Inventors: Kenneth Brown, Inver Grove Heights, MN (US); Ehab Hamed, Lexington, MA (US); Alan Harris, New York City, NY (US); Gary Hattersley, Stow, MA (US); Joan Moseman, Lake Elmo, MN (US); Jamal Saeh, Bemont, MA (US); Lisa Dick, Afton, MN (US)

(73) Assignees: Radius Health, Inc., Boston, MA (US); Kindeva Drug Delivery L.P., St. Paul, MN (US)

(21) Appl. No.: 17/395,516

(22) Filed: Aug. 6, 2021

Related U.S. Application Data
(63) Continuation of application No. PCT/IB2020/051699, filed on Feb. 27, 2020.
(60) Provisional application No. 62/812,140, filed on Feb. 28, 2019.

(51) Int. Cl. A61K 38/29; A61K 33/30; A61K 9/70; A61K 47/02; A61P 19/10

(52) U.S. Cl. CPC A61K 38/29; A61K 33/30; A61P 19/10; A61K 47/02; A61K 9/7023

(57) ABSTRACT

Provided herein are methods for treating osteoporosis and increasing bone mass density, that includes administering once a day, for about 5 minutes, a transdermal patch loaded with about 300 μg of abaloparatide, and ZnCl₂ at a molar ratio of 2.2:1 of ZnCl₂:abaloparatide. Also provided are the single-use transdermal patches loaded with about 300 μg of abaloparatide, and ZnCl₂ at a molar ratio of 2.2:1 of ZnCl₂:abaloparatide.

### RELATED APPLICATIONS

This application claims priority to U.S. Provisional Patent Application No. 62/812,140, filed on Feb. 28, 2019, the entire contents of which is expressly incorporated herein by this reference.

### TECHNICAL FIELD

The technical field is treatment of osteoporosis and other disorders, e.g., fracture repair, with a trans dermal formulation of abaloparatide.

### BACKGROUND

Abaloparatide, a human parathyroid hormone related peptide [PTHrP(1-34)] analog, is FDA approved as a once daily subcutaneous 80 mcg injection for postmenopausal women with osteoporosis at a high risk for fracture. Provided herein is an alternative to daily self-injection of abaloparatide without compromising the safety and efficacy of treatment with abaloparatide.

### SUMMARY OF THE INVENTION

Abaloparatide is a synthetic PTHrP analogue having the amino sequence:

SEQ ID NO: 1
Ala-Val-Ser-Glu-His-Gln-Leu-Leu-His-Asp-Lys-Gly-
Lys-Ser-Ile-Gln-Asp-Leu-Arg-Arg-Arg-Glu-Leu-Leu-
Glu-Lys-Leu-Leu-Aib-Lys-Leu-His-Thr-Ala-NH2

(TYMLOS abaloparatide injection label). Abaloparatide has shown potent anabolic activity with decreased bone resorption, less calcium-mobilizing potential, and improved room temperature stability. Subcutaneous administration of 80 μg abaloparatide has been shown to significantly reduce incidences of new vertebral, non-vertebral, major osteoporotic and clinical fractures. Subcutaneous abaloparatide administration has also been shown to improve bone mineral density (BMD) and/or trabecular bone score (TBS) of treated subjects at the lumbar spine, total hip, and femoral neck.

Trans dermal administration of abaloparatide is an attractive alternative to subcutaneous administration due to its less invasive nature. In particular, it might be advantageous in some contexts to develop transdermal abaloparatide administrations that are substantially bioequivalent or bioequivalent to the subcutaneous (SC) abaloparatide administration in order to benefit from its proven SC efficacy.

Disclosed herein are formulations, delivery devices, and dosing regimens that allow transdermal delivery of abaloparatide, providing equivalent benefits to the currently-available self-injection delivery option for abaloparatide.

In one aspect, a method for treating osteoporosis in a subject in need thereof is provided. The method includes administering daily a trans dermal patch applied to the subject's thigh for about 5 minutes, the patch comprising: 300 μg of abaloparatide, and ZnCl₂, at a molar ratio of 2.2:1 of ZnCl₂:abaloparatide, wherein the subject is treated for osteoporosis.

In another aspect, a method of increasing bone mass density (BMD) in a subject in need thereof is provided. The method includes administering daily a trans dermal patch applied to the subject's thigh for about 5 minutes, the patch comprising: 300 μg of abaloparatide, and ZnCl₂, at a molar ratio of 2.2:1 of ZnCl₂:abaloparatide, wherein the subject achieves at least a 5% increase in BMD by 6 months.

In another aspect, a once-daily trans dermal system for the delivery of abaloparatide is provided that includes a plurality of single-use trans dermal patches, each loaded with about 300 μg of abaloparatide, and ZnCl₂ at a molar ratio of 2.2:1 of ZnCl₂:abaloparatide; and instructions to administer one of the trans dermal patches once daily to the thigh for about 5 minutes. In some embodiments, the once-daily trans dermal system further includes a multi-use applicator. In some embodiments, the once-daily trans dermal system further includes a plurality of single use applicators.

In yet another aspect, a method for transdermally delivering abaloparatide to a subject in an amount bioequivalent to subcutaneous injection of 80 mcg abaloparatide is provided. The method includes administering daily a trans dermal patch applied to the subject's thigh for about 5 minutes, the patch comprising: 300 μg of abaloparatide, and ZnCl₂, at a molar ratio of 2.2:1 of ZnCl₂:abaloparatide, wherein an amount of abaloparatide is transdermally delivered to the subject that is bioequivalent to subcutaneous injection of 80 mcg abaloparatide.

In another aspect, a method for treating osteoporosis in a subject in need thereof is provided that includes administering daily a trans dermal patch applied to the subject's thigh for about 5 minutes, the patch comprising: 300 μg of abaloparatide, and one or more pharmaceutically acceptable zinc salts, at a molar ratio of 2.2:1 of the pharmaceutically acceptable zinc salts to abaloparatide, wherein the subject is treated for osteoporosis. In one embodiment the one or more pharmaceutically acceptable zinc salts includes zinc chloride. In another embodiment the one or more pharmaceutically acceptable zinc salts is or includes zinc acetate.

In another aspect, a method of increasing bone mass density (BMD) in a subject in need thereof is provided that includes administering daily a trans dermal patch applied to the subject's thigh for about 5 minutes, the patch comprising: 300 μg of abaloparatide, and one or more pharmaceutically acceptable zinc salts, at a molar ratio of 2.2:1 of pharmaceutically acceptable zinc salts to abaloparatide, wherein the subject achieves at least a 5% increase in BMD by 6 months. In one embodiment the one or more pharmaceutically acceptable zinc salts includes zinc chloride. In another embodiment the one or more pharmaceutically acceptable zinc salts is or includes zinc acetate.

In yet another aspect, a once-daily trans dermal system for the delivery of abaloparatide is provided that includes a plurality of single-use trans dermal patches, each loaded with about 300 μg of abaloparatide, and one or more pharmaceutically acceptable zinc salts at a molar ratio of 2.2:1 of pharmaceutically acceptable zinc salts:abaloparatide, and instructions to administer one of said trans dermal patches once daily to the thigh for about 5 minutes. In one embodiment the one or more pharmaceutically acceptable zinc salts includes zinc chloride. In another embodiment the one or more pharmaceutically acceptable zinc salts is or includes zinc acetate. In another embodiment the one or more pharmaceutically acceptable zinc salts is or includes zinc acetate, zinc carbonate, zinc chloride, zinc gluconate, zinc oxide, zinc sulfate and combinations thereof. In one embodiment the system includes a multi-use applicator. Alternatively, the once-daily trans dermal system includes a plurality of single use applicators.

In yet another aspect, a method for transdermally delivering abaloparatide to a subject in an amount bioequivalent to subcutaneous injection of 80 mcg abaloparatide is provided. The method includes administering daily a trans dermal patch applied to the subject's thigh for about 5 minutes, the patch comprising: 300 μg of abaloparatide, and one or more pharmaceutically acceptable zinc salts, at a molar ratio of 2.2:1 of pharmaceutically acceptable zinc salt:abaloparatide, wherein an amount of abaloparatide is trans dermally delivered to the subject that is bioequivalent to subcutaneous injection of 80 mcg abaloparatide.

In one embodiment the one or more pharmaceutically acceptable zinc salts includes zinc chloride. In another embodiment the one or more pharmaceutically acceptable zinc salts is or includes zinc acetate. In another embodiment the one or more pharmaceutically acceptable zinc salts is or includes zinc acetate, zinc carbonate, zinc chloride, zinc gluconate, zinc oxide, zinc sulfate and combinations thereof.

In another aspect, an aqueous formulation suitable for coating a trans dermal patch is provided wherein the aqueous formulation comprises 300 μg of abaloparatide, zinc at a molar ratio of 2.2:1 of Zn:abaloparatide, and hydrochloric acid. In some embodiments, the pH of the aqueous formulation is between about 4.5 and about 5. In some embodiments, the pH is between about 4 and about 4.75. In some embodiments, the pH is about 4.5. In some embodiments, the pH is less than 4.75. In some embodiments the mole ratio of HCl to zinc chloride is about 0.025, at least about 0.025, between about 0.02 to about 0.1, or between about 0.02 and about 0.07.

In yet another aspect, a trans dermal system for the delivery of abaloparatide is provided. The system includes an abaloparatide trans dermal patch made by coating a plurality of microprojections defined by a surface of a trans dermal patch with the above aqueous formulation; and instructions to administer one of said trans dermal patches once daily to the thigh for about 5 minutes.

### BRIEF DESCRIPTION OF THE DRAWINGS

FIG. 1A and FIG. 1B are a graph of plasma concentrations over time and a table of pharmacokinetic parameters, respectively, for Cohort 1, Formula A at 100 mcg, 150 mcg and 200 mcg doses.

FIG. 2A and FIG. 2B are a graph of plasma concentrations over time and a table of pharmacokinetic parameters, respectively, for Cohort 2, Formula B at 100 mcg, 150 mcg and 200 mcg doses.

FIG. 3A and FIG. 3B are a graph of plasma concentrations over time and a table of pharmacokinetic parameters, respectively, for Cohort 3, Formula C at 100 mcg, 150 mcg and 200 mcg doses.

FIG. 4A and FIG. 4B are a graph of plasma concentrations over time and a table of pharmacokinetic parameters, respectively, for Cohort 4.

FIG. 5A and FIG. 5B are a graph of plasma concentrations over time and a table of pharmacokinetic parameters, respectively, for Cohort 5.

FIG. 6A-6E are graphs of plasma concentrations for Cohort 6 in linear scale (FIG. 6A) and semi-log scale (FIG. 6B); and tables of pharmacokinetic parameters for Cohort 6 (FIG. 6C), a within cohort comparison (FIG. 6D), and relative bioavailability parameters between doses, application sites, and wear times (FIG. 6E).

FIG. 7A and FIG. 7B are graphs of plasma concentrations over time for Cohort 7 in linear and semi-log scale, respectively.

### DETAILED DESCRIPTION

Provided herein is a safe, effective, and pain-free alternative to daily self-injection of abaloparatide in the form of a trans dermal device. Application time is only about 5 minutes, yet surprisingly provides a bioequivalent amount of abaloparatide to that achieved with self-injection. The device is a once-daily trans dermal patch that includes 300 μg of abaloparatide disposed on the patch with a release modulating agent, ZnCl₂. This release modulating agent is present on the trans dermal device at a molar ratio of 2.2:1 of ZnCl₂:abaloparatide. That delivery of an effective and safe amount can be achieved with a residence time of about 5 minutes for the patch is unexpected and surprising as demonstrated in the exemplification.

### Definition and Abbreviations

The terms “ZnCl₂” and “zinc chloride” are used interchangeably, and refer to the molecule of zinc chloride including all hydrates and solvates.

The term “pharmaceutically acceptable zinc salts” refers to pharmaceutically acceptable zinc salts, including solvates and hydrates that are generally recognized, by qualified experts, to be safe under the intended conditions of use (e.g., GRAS as recognized by the FDA). Zinc salts include zinc acetate, zinc carbonate, zinc chloride, zinc gluconate, zinc oxide and zinc sulfate. While the zinc in the zinc salts may (or may not) disassociate from the chloride in water and be dried to include forms that are different from the original compound, it is still referred to as zinc salt for ease of reference and clarity.

In this application, the amount of pharmaceutically acceptable zinc salt to abaloparatide is described as a mole ratio which is represented herein as “M” unless stated otherwise. For example, a coating solution described as 2.2 M ZnCl₂ indicates a mole ratio of ZnCl₂ to abaloparatide of 2.2:1. The molar ratio is determined, e.g., by calculation of the ratio of ZnCl₂ to abaloparatide added to the coating solution on a molar basis. While the zinc in ZnCl₂ may disassociate from the chloride in water and be dried to include various hydrates, solvates and other forms, the amount of zinc on a molar basis will not substantially differ from that added, and as a result, is still referred to as ZnCl₂ in the transdermal patch.

The terms “transdermal device” and “patch” are used interchangeably in this application. Suitable trans dermal devices include devices having an array of microstructures that pierce the stratum corneum when pressed against the skin to deliver an agent to the tissues below. Microneedles in the form of micro-blades or microstructures (e.g., as disclosed in WO2017/184355 published 26 Oct. 2017 and filed as PCT/US2017/026462 on 6 Apr. 2017), pierce the stratum corneum upon application of force, making a plurality of tiny openings or slits which serve as passageways through which abaloparatide can be delivered to the body. Alternatively, the microneedles can be hollow to provide a liquid flow path from a reservoir to the microneedles. These trans dermal devices can be deployed with a single-use applicator or an application capable of being used multiple times. The trans dermal patch or device can be any of the patches described herein, or described in International Application Nos. PCT/US2016/056196, filed on Oct. 8, 2016 and published as WO2017/062922, PCT/2017/026462, filed Apr. 6, 2017 and published as WO 2017/184355, or PCT/US2016/055924, filed Oct. 7, 2016 and published as WO2017/062727. The entire content of which as expressly incorporated herein by this reference.

The term “applicator” refers to a device for applying a trans dermal device or patch to the skin with sufficient force for the microneedle array to pierce the stratum corneum and deliver abaloparatide to the subject.

The abbreviations employed in this application are provided in Table 1: List of Abbreviations.

TABLE 1
List of Abbreviations

| | |
|---|---|
| ABL | Abaloparatide |
| %AUCextrap | Extrapolated portion of AUC0-inf from Tlast to |
| | infinity |
| AUC | Area under the curve |
| AUC0-inf | Area under the curve to infinity |
| AUC0-t | Area under the plasma concentration-time curve |
| BLQ | Below the limit of quantification |
| BMD | Bone mineral density |
| CI | Confidence interval |
| CL/F | Apparent clearance |
| Clast | Last quantifiable concentration |
| Cmax | Maximal plasma concentration |
| CV% | Coefficient of variation |
| Frel | Relative bioavailability in % |
| h | Hour |
| hPTH | Human parathyroid hormone |
| hPTHrP | Human parathyroid-related peptide |
| Kel | Apparent terminal elimination rate constant |
| ln | natural-log |
| LS | Least-square |
| Max | Maximum |
| Mean | Arithmetic mean |
| Min | Minimum |
| min | Minute |
| N | Sample size, number of observation |
| PK | Pharmacokinetic (s) |
| PTH | Parathyroid hormone |
| PTHrP | Parathyroid hormone-related peptide |
| SC | Subcutaneous injection |
| SD | Standard deviation |
| t1/2 | Apparent terminal elimination half-life |
| TD | Trans dermal delivery system |
| Tlast | Time of the last quantifiable concentration |
| Tmax | Time to maximal concentration |
| μg or mcg | micrograms (used interchangeably) |
| Vz/F | Apparent volume of distribution |

### EXEMPLIFICATION

An open-label, partially randomized, single-dose crossover, pilot PK, safety and tolerability study was conducted in healthy postmenopausal women to select the formulation, dose, application site, and wear time for trans dermal abaloparatide (abaloparatide-TD) with a PK profile comparable to TYMLOS abaloparatide subcutaneous injection (abaloparatide-SC).

Formulations used in the Cohorts are shown in Table 2: Study Formulations.

TABLE 2
Study Formulations

| | | | Study |
|---|---|---|---|
| Formulation | Doses | Formulation Components | Cohorts |
| A | 100, 150, 200, 260 μg | Abaloparatide:ZnCl₂ 1:0.7 molar ratio | 1, 4, 5 |
| | 2 × 150 μg | | |
| B | 100, 150, 200 μg | Abaloparatide polyethylene glycol (PEG) | 2 |
| C | 100, 150, 200 μg | Abaloparatide:ZnCl₂ 1:0.7 molar ratio plus PEG | 3, 4 |
| W | 200, 300, 400 μg | Abaloparatide:ZnCl₂ 1:0.71:2.2 molar ratio | 5, 6 |
| | 2 × 200 μg | | |
| X | 200 μg | Abaloparatide:ZnAc 1:0.7 molar ratio | 5 |
| W-1 | 300 μg | Abaloparatide:ZnCl₂ 1:2.2 molar ratio, HCl | 7 |
| Reference: | 80 μg | Abaloparatide | 1, 2, 3, 6, 7 |
| SC | | | |

The formulated patch is made by coating with the coating solution in one or multiple coating iterations and then drying the patch or allowing the patch to dry to a fairly constant weight.

Formulation A 0.7 M ZnCl₂ abaloparatide coating solution (Table 3.1).

TABLE 3.1
Formulation A: 0.7 M ZnCl₂ abaloparatide coating solution

| Component | Weight (about) % |
|---|---|
| Abaloparatide | 45.11 |
| Zinc Chloride, USP (ZnCl₂) | 0.89 (approx. 0.7 mole ratio to |
| | abaloparatide) |
| Sterile Water for Injection, USP | 54.00 |
| Total | 100 |

Formulation A 0.7 M ZnCl₂ abaloparatide formulation on patch ready to use (after drying) (Table 3.2).

TABLE 3.2
Formulation A:
0.7 M ZnCl₂ abaloparatide formulation on patch ready to use
(after drying)

| Component | Weight (about) % |
|---|---|
| Abaloparatide | 98.07 |
| Zinc Chloride, USP (ZnCl₂) | 1.93 |
| Total | 100 |

Formulation B: abaloparatide PEG coating solution (Table 3.3).

TABLE 3.3
Formulation B: abaloparatide PEG coating solution

| Component | Weight (about) % |
|---|---|
| Abaloparatide | 40.5 |
| Polyethylene Glycol 3350NF | 14.5 |
| Sterile Water for Injection, USP | 45.00 |
| Total | 100 |

Formulation B: abaloparatide PEG formulation on patch ready to use (after drying) (Table 3.4).

TABLE 3.4
Formulation B:
abaloparatide PEG formulation on patch ready to use
(after drying)

| Component | Weight (about) % |
|---|---|
| Abaloparatide | 73.64 |
| Polyethylene Glycol 3350NF | 26.36 |
| Total | 100 |

Formulation C: Abaloparatide: ZnCl₂ 1:0.7 molar ratio plus PEG coating solution (Table 3.5).

TABLE 3.5
Formulation C:
Abaloparatide:ZnCl₂ 1:0.7 molar ratio plus PEG
coating solution

| Component | Weight (about) % |
|---|---|
| Abaloparatide | 34.84 |
| Polyethylene Glycol 3350NF | 12.47 |
| Zinc Chloride, USP | 0.69 |
| Sterile Water for Injection, USP | 52 |
| Total | 100 |

Formulation C: Abaloparatide:ZnCl₂ 1:0.7 molar ratio plus PEG formulation on patch ready to use (after drying) (Table 3.6).

TABLE 3.6
Formulation C:
Abaloparatide:ZnCl₂ 1:0.7 molar ratio plus PEG
coating solution

| Component | Weight (about) % |
|---|---|
| Abaloparatide | 72.58 |
| Polyethylene Glycol 3350NF | 25.98 |
| Zinc Chloride, USP | 1.44 |
| Total | 100 |

Formulation W: 2.2M ZnCl₂ abaloparatide coating solution (Table 3.7).

TABLE 3.7
Formulation W:
2.2 M ZnCl₂ abaloparatide coating solution

| Component | Weight (about) % |
|---|---|
| Abaloparatide | 35.78 |
| Zinc Chloride, USP (ZnCl₂) | 2.22 (approx. 2.2 mole ratio |
| | Zn to abaloparatide) |
| Sterile Water for Injection, USP | 62.00 |
| Total | 100 |

Formulation W: 2.2M ZnCl₂ abaloparatide formulation on patch ready to use (after drying) (Table 3.8).

TABLE 3.8
Formulation W:
2.2 M ZnCl abaloparatide formulation on patch
ready to use (after drying)

| Component | Weight (about) % |
|---|---|
| Abaloparatide | 94.16 |
| Zinc Chloride, USP (ZnCl₂) | 5.84 |
| Total | 100 |

Formulation W-1: 2.2M ZnCl₂ abaloparatide formulation with HCl on patch ready to use (after drying) (Table 3.8.1).

TABLE 3.8.1
Formulation W:
2.2 M ZnCl₂ abaloparatide formulation on patch
ready to use (after drying)

| Component | Weight (about) % |
|---|---|
| Abaloparatide | 93.85 |
| Zinc Chloride, USP (ZnCl₂) | 6.11 |
| HC1 | 0.04 |
| Total | 100.00 |

Formulation X: ZnAc abaloparatide coating solution (Table 3.9).

TABLE 3.9
Formulation X:
ZnAc abaloparatide coating solution

| Component | Weight (about) % |
|---|---|
| Abaloparatide | 40.62 |
| Zinc Acetate, USP | 1.38 (about 0.7 mole ratio to |
| | abaloparatide) |
| Sterile Water for Injection, USP | 58.00 |
| Total | 100 |

Formulation X: ZnAc abaloparatide formulation on patch ready for use (after drying) (Table 3.10).

TABLE 3.10
Formulation X:
ZnAc abaloparatide formulation on patch ready for
use (after drying)

| Component | Weight (about) % |
|---|---|
| Abaloparatide | 96.7 |
| Zinc Acetate, USP | 3.3 |
| Total | 100 |

### Transdermal System

Subjects received a single application of a trans dermal patch (500×550 patch; needles had a length of 500 μm and needle tips were spaced 550 μm from each other). Microneedle trans dermal patches coated with the formulations of abaloparatide were stored refrigerated at 2-8° C. At least one hour prior to use, the trans dermal patches in individual pouches were placed at room temperature. The area of a single patch with microneedles was typically about 1.26 cm². If two patches were used, they had a combined area of about 2.52 cm². The patch was applied by pushing the delivery device containing the patch to the skin at a force of, e.g., 15-25 newtons. The energy at impact to the patch upon delivery is delivered very quickly to the stratum corneum with a penetration time of less than, for example 50 milliseconds or even less than 10 milliseconds and energy sufficient to penetrate the stratum corneum.

### Pharmacokinetic Assessments

In Cohorts 1-5, a total of 10 venous blood samples were drawn from each subject in each treatment period to measure abaloparatide plasma concentrations at the following time points (clock starts from time of application/injection): 0 (pre-dose), 5, 10, 20, 30, and 60 minutes and 1.5, 2, 3, and 24 hours post-dose.

Beginning in Cohort 6, a total of 11 venous blood samples were drawn from each subject in each treatment period to measure abaloparatide plasma concentrations at the following time points: 0 (pre-dose), 5, 10, 20, 30, and 60 minutes and 1.5, 2, 3, 4, and 24 hours post-dose.

In Cohort 7, a total of 12 venous blood samples were drawn from each subject in each treatment period to measure abaloparatide plasma concentrations at the following time points( ) (pre-dose), 5, 10, 20, 30, and 60 minutes and 1.5, 2, 3, 4, 8, and 24 hours post-dose.

PK parameters of plasma abaloparatide were calculated using a validated Phoenix™ WinNonlin® 7. Summary tables and figures of abaloparatide in plasma were generated using a validated version of Phoenix™ WinNonlin® 7 or R Version 3.4.4. Inferential statistical analyses were performed using validated version of Phoenix™ WinNonlin® 7 (Average Bioequivalence Module).

The total dose of abaloparatide released from the patch was used for PK parameter calculation following abaloparatide-TD. The total released dose was calculated in the source dataset as: Total Released Dose (μg)=Initial Patch Content (μg)−Patch Residual Drug (μg)−Skin Swab Residual Drug (μg). The nominal dose of abaloparatide (i.e., 80 μg) was used for PK parameter calculations following abaloparatide-SC.

### Cohorts 1-3: Study Design

The study design of Cohorts 1 through 3 followed a 4 period Williams Latin Square Design in which an equal number of subjects in each cohort were randomized to 1 of the 4 treatment sequences. In this design, each subject received each of the 4 treatments in the cohort over the course of the 4 treatment periods (Table 4). All abaloparatide-TD formulations were applied to the periumbilical region of the abdomen for 15 minutes.

TABLE 4
Cohorts 1 to 3 Treatment Design
(All TD treatments used Applicator 1, Site-Abdomen, 15 minutes)
Cohort 1 (Formulation A)
Cohort 2 (Formulation B)
Cohort 3 (Formulation C)

| | Treatment Period | | | |
|---|---|---|---|---|
| Sequence | 1 | 2 | 3 | 4 |
| 1 | SC 80 μg | TD 200 μg | TD 100 μg | TD 150 μg |
| 2 | TD 100 μg | SC 80 μg | TD 150 μg | TD 200 μg |
| 3 | TD 150 μg | TD 100 μg | TD 200 μg | SC 80 μg |
| 4 | TD 200 μg | TD 150 μg | SC 80 μg | TD 100 μg |

Subjects were randomized to receive 1 of the 4 possible dosing sequences shown using equal allocation ratio in each cohort. This design was used for Cohorts 1, 2, and 3 with an evaluation period after each cohort. A different TD patch formulation was used for each cohort. Subjects were enrolled into 1 of 3 cohorts, with each cohort receiving a different TD formulated patch. Each treatment period was separated by a washout period of at least 7 days.

### Cohort 1 Results

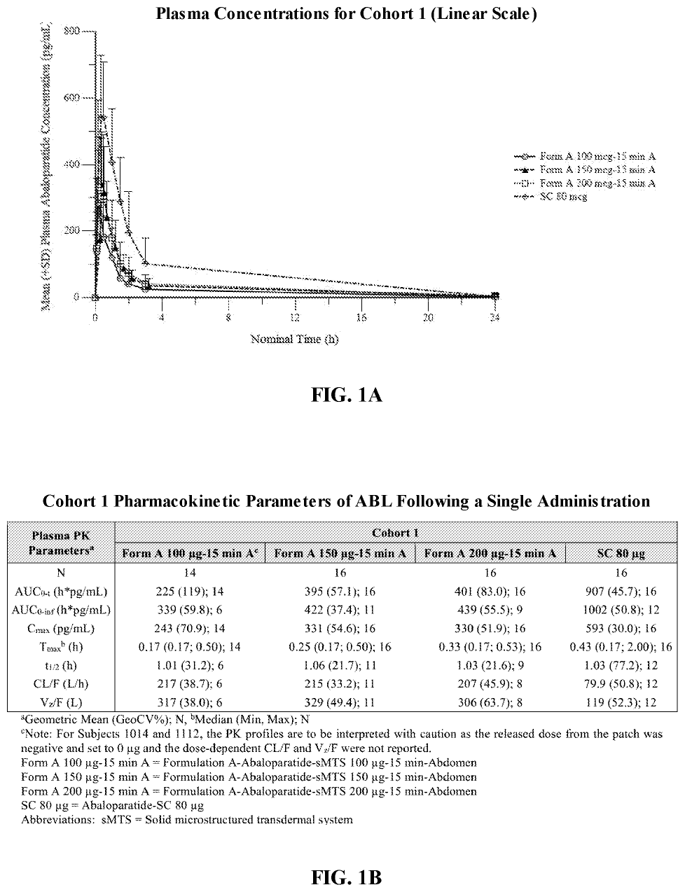

Referring to FIG. 1A and FIG. 1B, following a single dose of abaloparatide-TD Formulation A at 3 dose levels, geometric mean plasma abaloparatide AUC and Cₘₐₓ increased from the 100 to 150 μg dose levels and remained stable from the 150 to 200 μg dose levels. Systemic exposure parameters following abaloparatide-TD Formulation A achieved 25 to 44% of abaloparatide-SC AUC and 41 to 56% of abaloparatide-SC Cₘₐₓ.

Assuming a dose proportional increase in systemic exposure, the observed CL/F suggested that a patch of 350 μg would be required to achieve similar systemic exposure to the SC 80 μg treatment. Given that the systemic exposure (Cₘₐₓ and AUC₀₋ₜ) appeared to plateau between 150 and 200 μg TD, the likelihood of successfully matching the target 80 μg SC results with Formulation A appeared low.

Based on AUC₀₋ₜ, AUC_{0-inf}, and Cₘₐₓ values, the relative bioavailability of abaloparatide following a single administration of abaloparatide-TD Formulation A at dose levels of 100, 150 and 200 μg was 44.1 to 73.6% lower than that of abaloparatide-SC and the 90% CI were outside the 80 to 125% acceptance criteria for similarity for all comparisons.

### Cohort 2 Results

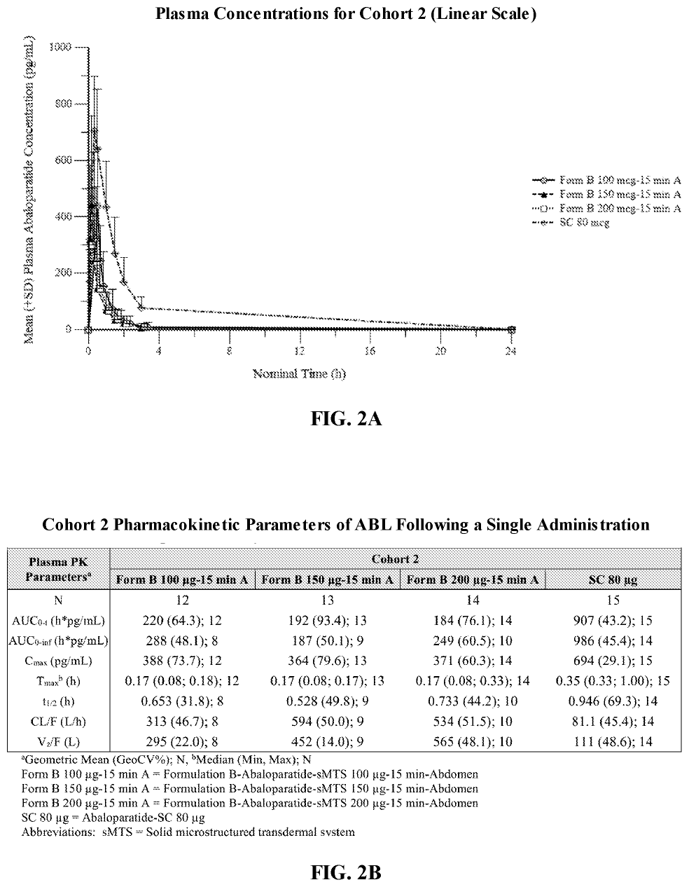

Referring to FIG. 2A and FIG. 2B, following abaloparatide-TD Formulation B, mean plasma abaloparatide concentrations were similar across the 100 to 200 μg dose levels. Mean plasma abaloparatide concentrations were lower following abaloparatide-TD Formulation B for all dose levels compared to abaloparatide-SC.

Systemic exposure parameters following abaloparatide-TD Formulation B achieved 19 to 29% of abaloparatide-SC AUC and 52 to 56% of abaloparatide-SC Cₘₐₓ. Based on AUC₀₋ₜ, AUC_{0-inf}, and Cₘₐₓ values, the relative bioavailability of abaloparatide following a single administration of abaloparatide-TD Formulation B at dose levels of 100, 150 and 200 μg was 46.6 to 83.0% lower than that of abaloparatide-SC and the 90% CI were outside the 80 to 125% acceptance criteria for similarity for all comparisons.

### Cohort 3 Results

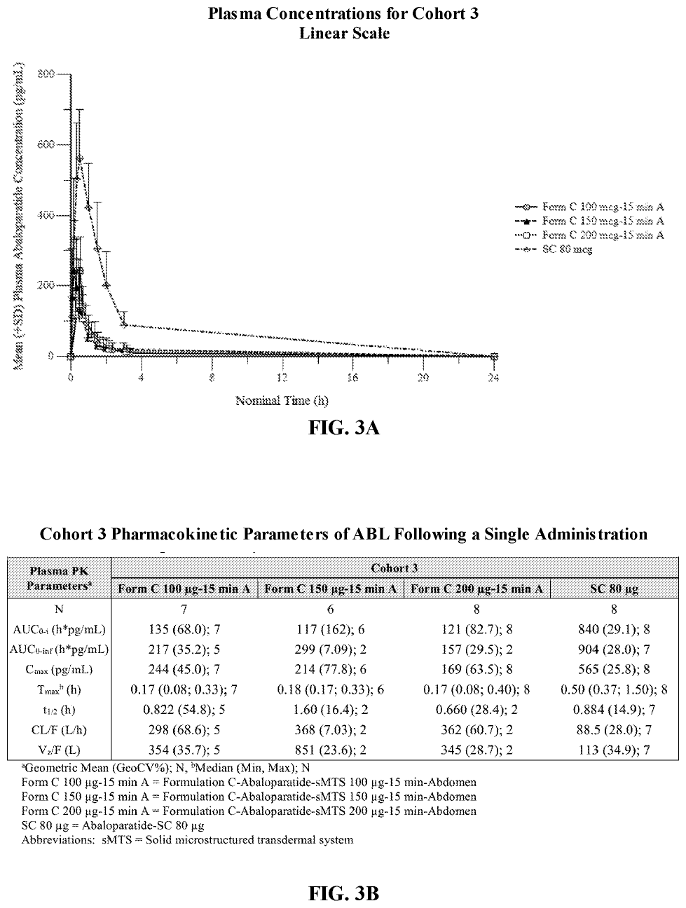

Referring to FIG. 3A and FIG. 3B, following abaloparatide-TD Formulation C, mean plasma abaloparatide concentrations were similar across the 100 to 200 μg dose levels. Mean plasma abaloparatide concentrations were lower following abaloparatide-TD Formulation C for all dose levels compared to abaloparatide-SC.

Systemic exposure parameters following abaloparatide-TD Formulation C achieved 14 to 33% of abaloparatide-SC AUC and 30 to 43% of abaloparatide-SC Cₘₐₓ. And based on AUC₀₋ₜ, AUC_{0-inf}, and Cₘₐₓ values, the relative bioavailability of abaloparatide following a single administration of abaloparatide-TD Formulation C at dose levels of 100, 150 and 200 μg was 59.1 to 86.9% lower than that of abaloparatide-SC and the 90% CI were outside the 80 to 125% acceptance criteria for similarity for all comparisons.

### Summary of Cohorts 1 to 3

Overall, for Cohorts 1 to 3, the AUC₀₋ₜ, AUC_{0-inf}, and Cₘₐₓ of abaloparatide following a single administration of abaloparatide-TD Formulations A, B or C at dose levels of 100, 150 or 200 μg were lower than those observed for 80 μg abaloparatide-SCₘₐₓ The systemic exposure (Cₘₐₓ and AUC₀₋ₜ) of abaloparatide-TD Formulation A increased from the 100 to 150 μg dose levels and appeared to plateau between 150 and 200 μg. As for Formulations B and C (Cohort 2 and 3), the systemic exposure did not increase with increasing dose and appeared to have already plateaued at 100 μg. Assuming a dose-proportional increase in systemic exposure, a dose of 350 μg of Formulation A would be required to match the systemic exposure of 80 μg abaloparatide-SC.

### Cohorts 4 and 5

In Cohort 4, subjects received abaloparatide-TD Formulation A and Formulation C at 200 μg dose applied to the ventral midline of the thigh with a wear time of 15 minutes. In Cohort 4, treatment period 3, each subject was given study medication through simultaneous application of two abaloparatide-TD 150 μg patches in separate quadrants of the abdomen (Applicator 1). The abaloparatide-TD microneedle patches were applied for 15 minutes, and each treatment period was separated by a washout period of at least 3 days.

Cohort 5 evaluated four formulations of abaloparatide-TD. Each patch contained a dose of either 200 μg or 260 μg of abaloparatide, applied as a single patch administration to the thigh, or a simultaneous double patch application of 200 μg applied to the ventral midline of thigh with a different patch applicator (Applicator 2). All abaloparatide-TD formulations were applied for 15 minutes. A summary of the design for Cohorts 4 and 5 is shown in Table 5.

TABLE 5
Treatments for Cohorts 4 and 5
Cohort 4, all TD treatments used Applicator 1, 15 Minutes
Cohort 5, all TD treatments used Applicator 2, 15 Minutes

| Sequence | Period 1 | Period 2 | Period 3 | Period 4 |
|---|---|---|---|---|
| 4 | Form A, | Form C, | Form A, | |
| | 200 μg, | 200 μg, | 2 × 150 μg, | |
| | Thigh | Thigh | Abdomen | |
| 5 | Form W, | Form X, | Form A, | Form W, |
| | 200 μg, | 200 μg, | 260 μg, | 2 × 200 μg, |
| | Thigh | Thigh | Thigh | Thigh |

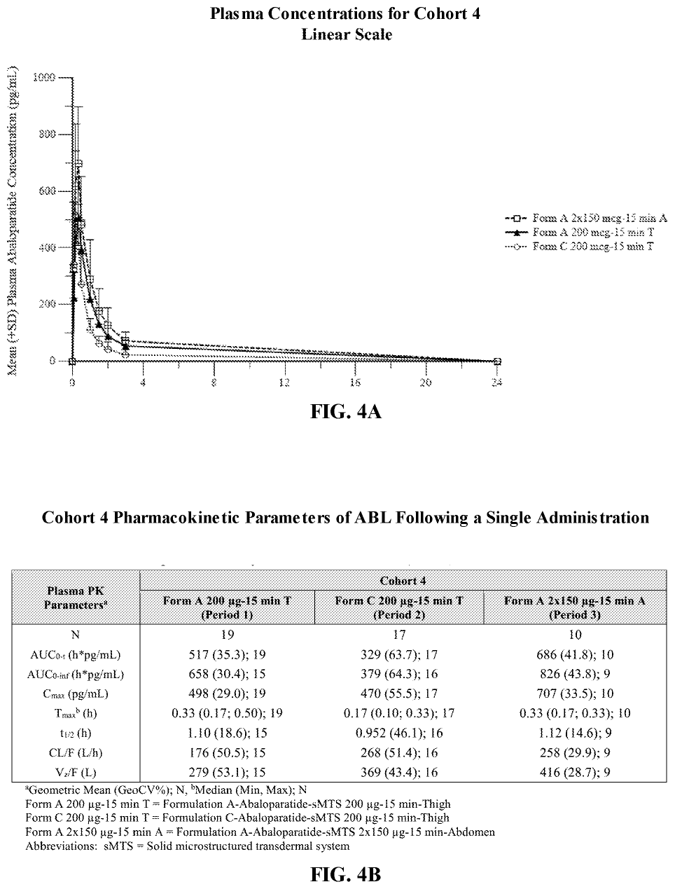

Referring to FIG. 4A and FIG. 4B, mean plasma abaloparatide concentrations were higher following abaloparatide-TD Formulation A 200 μg applied to the thigh and 2×150 μg applied to the abdomen treatments compared to abaloparatide-TD Formulation C 200 μg. Mean plasma abaloparatide concentrations were higher with the double patch Formulation A2×150 μg applied to the abdomen compared with the 200 μg single patch applied to the thigh. Based on AUC₀₋ₜ, AUC_{0-inf} and Cₘₐₓ values, the relative bioavailability of abaloparatide following a single administration of abaloparatide-TD Formulations A and Cat dose levels of 200 μg and 2×150 μg was 20.6 to 65.5% lower than that of abaloparatide-SC, except for similar Cₘₐₓ (geometric ratio of 108%) achieved with Formulation A 2×150 μg.

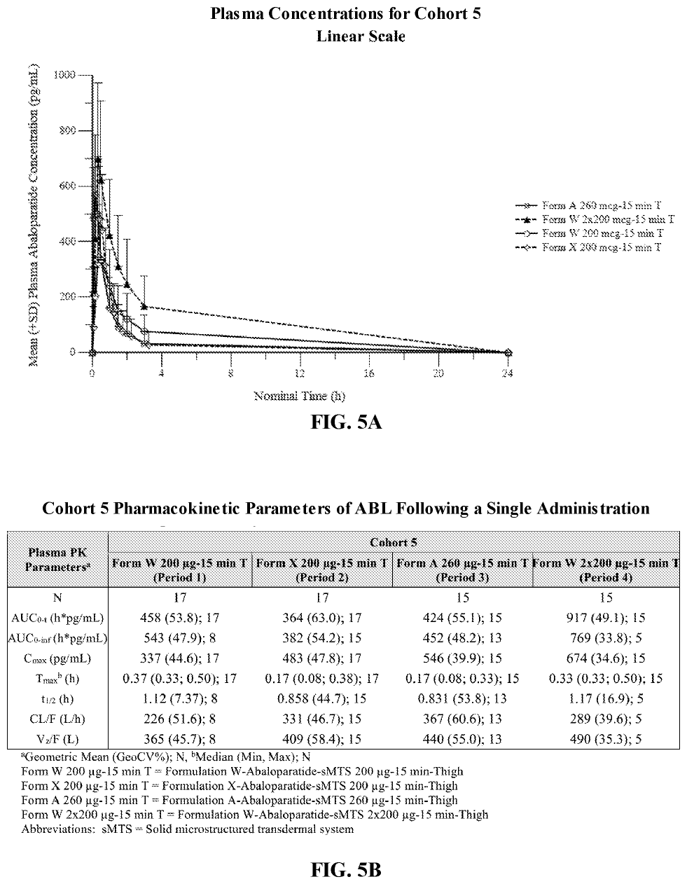

Referring to FIG. 5A and FIG. 5B, the abaloparatide-TD Formulation W 2×200 μg double patches achieved the highest mean plasma abaloparatide concentrations followed by Formulation W 200 μg and then Formulation A 260 μg and Formulation X 200 μg with similar concentration levels. Based on AUC₀₋ₜ, AUC_{0-inf} and Cₘₐₓ values, the relative bioavailability of abaloparatide following a single administration of abaloparatide-TD Formulations A, W and X at dose levels of 200 and 260 μg was 26.3 to 63.3% lower than that of abaloparatide-SC, except for similar Cₘₐₓ (geometric ratio of 83.2%) achieved with Formulation A 260 μg.

Formulation W 2×200 μg applied 15 minutes to the thigh (Cohort 5) was the most similar to 80 μg abaloparatide SC with AUC₀₋ₜ and Cₘₐₓ values achieving 96.2 and 103% of those of abaloparatide SC, respectively. The double patch application Formulation A 2×150 μg (300 μg total) (Cohort 4) and Formulation W 2×200 μg (400 μg total) (Cohort 5) increased the systemic exposure in a dose-proportional manner compared to a single patch application of 200 μg. Although the application site was different, the AUC₀₋ₜ was increased by ˜33% and Cₘₐₓ by ˜42% with Formulation A after increasing the dose by 50% using double patches (i.e., 2×150 μg vs. 200 μg), by increasing the dose released from the patches, which could not be achieved via a greater patch loading dose.

The best performing abaloparatide formulation in Cohorts 1-5 was Formulation W 2×200 μg applied to the thigh for 15 minutes. And dose proportional systemic exposure for Formulation W was achieved by increasing the number of patches. Doses, application sites and wear times for Formulation W were evaluated further in Cohort 6.

### Cohort 6

Cohort 6 evaluated 3 wear times and 3 doses of abaloparatide-TD, each applied as a single patch administration to the thigh or abdomen using applicator 2. Subjects were randomized for Treatment Periods 1 and 2 to receive abaloparatide-TD 400 μg and abaloparatide-TD 300 μg in a crossover design, applied to the thigh for a 15 minute wear time. For Treatment Periods 3 and 4, subjects received abaloparatide-TD 400 μg applied to the thigh, either for a 5 minute or a 30 minute wear time, respectively. Based on previous cohorts, additional Treatment Periods (5A and/or 5B) were considered, but only Period 5B was evaluated. Subjects in Treatment Period 5B received the reference SC dose (abaloparatide-subcutaneous [SC] 80 μg). For Treatment Period 6, subjects received abaloparatide-TD 300 μg applied to one of the upper quadrants the abdomen for a 15 minute wear time. A summary of the design for Cohort 6 is shown in Table 6.

TABLE 6
Treatments for Cohort 6
All TD treatments used Formulation W, Applicator 2

| Se- | | | | | Period | |
|---|---|---|---|---|---|---|
| quence | Period 1 | Period 2 | Period 3 | Period 4 | 5B | Period 6 |
| 1 | 400 μg, | 300 μg, | 400 μg, | 400 μg, | SC | 300 μg, |
| | Thigh, | Thigh, | Thigh, | Thigh, | 80 μg | Abdomen, |
| | 15 Minutes | 15 Minutes | 5 | 30 | | 15 |
| 2 | 300 μg, | 400 μg, | Minutes | Minutes | | minutes |
| | Thigh, | Thigh, | | | | |
| | 15 Minutes | 15 Minutes | | | | |

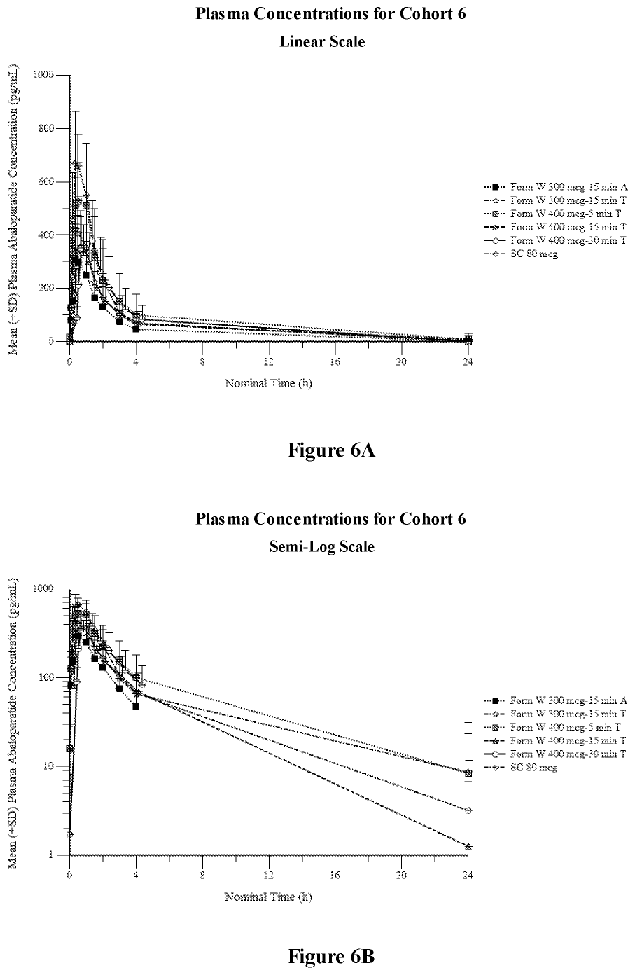

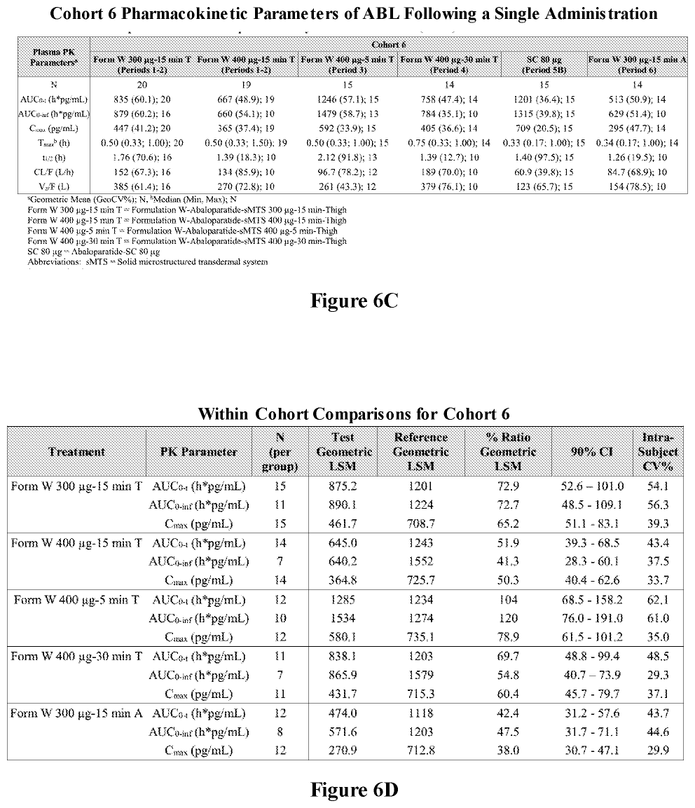

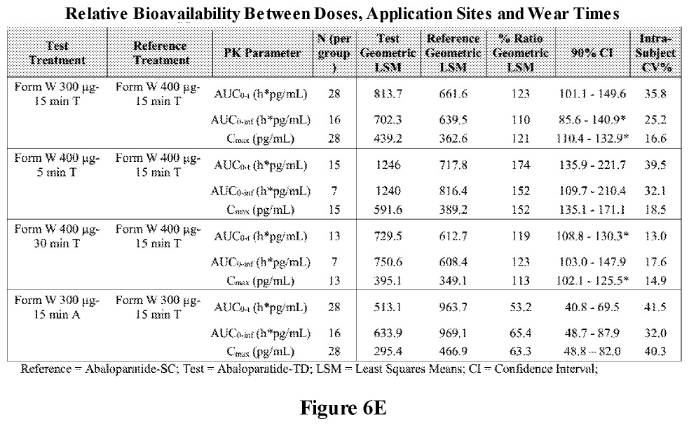

Referring to FIG. 6A-6C, abaloparatide was absorbed with mean tmax ranging from 20 to 60 minutes for TD treatments, compared to 30 minutes for SCₘₐₓ Formulation W 300 μg produced 25% to 40% higher abaloparatide Cₘₐₓ and AUC than the Formulation W 400 μg (Thigh, 15 minutes). Similar to assessments in previous cohorts, Formulation W 300 μg application to the thigh produced 60% to 80% higher abaloparatide Cₘₐₓ and AUC than application to the abdomen.

As shown in FIG. 6C, formulation W 400 μg applied for 5 minutes to the thigh provided similar AUC and Cₘₐₓ compared to the SC 80 μg within the same cohort of subjects (4 to 20% higher AUC and 21.1% lower Cₘₐₓ).

However, compared to the SC 80 μg data pooled across 4 studies, FIG. 6E shows Formulation W 400 μg applied for 5 minutes to the thigh provided 45 to 59% higher AUC and 3.7% lower Cₘₐₓ). When the Formulation W 300 μg patch was applied to the abdomen, the systemic exposure achieved was 34.6 to 46.8% lower than that of achieved for same patch applied to the thigh. For the Formulation W 400 μg patch, the highest systemic exposure levels were reached when the patch was applied for the shortest tested wear time with AUC₀₋ₜ, AUC_{0-inf} and Cₘₐₓ values 52 to 74% higher after 5 minutes than after 15 minutes. Increasing the wear time to 30 minutes did not seem to have any impact on systemic exposure, with similar AUC₀₋ₜ, AUC_{0-inf} and Cₘₐₓ values (13% to 23% higher geometric means) after 15 and 30 minutes.

Referring to FIG. 6D, for Formulation W 400 μg applied to the thigh, AUC_{5 min}>AUC_{30 min}>AUC_{15 min} (relative bioavailability compared to within-cohort SC was 104%-121%, 55%-70%, and 41%-52%, respectively). Formulation W 300 μg (Thigh, 15 minutes) had a 73% relative bioavailability compared to within-cohort SC, better than 400 μg (Thigh, 15 minutes). Formulation W 300 μg (Thigh, 5 minutes) also had a 96%-99% relative bioavailability compared to pooled SC treatments.

These results suggested that wear time has a significant factor in the availability of abaloparatide, although the relationship between wear time and systemic exposures was unexpected. For example, at a common dose of 400 μg, the shortest wear time of 5 minutes produced higher systemic exposure than the wear time of 30 minutes, which was slightly greater than the systemic exposure for a wear time of 15 minutes. The reason for this unusual rank order based on wear time is not clear. Decreasing the wear time from 15 to 5 minutes essentially doubled the systemic exposure. However, increasing the wear time to 30 minutes had only a small impact on systemic exposure. The thigh appeared to be a better application site compared to the abdomen, as there was a ˜50 to 60% increase in systemic exposure following administration at that site. Overall, administering a 400 μg patch of Formulation W for 5 minutes to the thigh produced systemic exposures comparable to an 80 μg SC administration. Although for the same 15-minute wear time and application to the thigh, the systemic exposure for Formulation W 300 μg was higher than the systemic exposure for Formulation W 400 μg.

Based on these results, Cohort 7 was designed to explore four different wear times (5 min, 15 min, 30 min, and 24 hours) for Formulation W-1 300 μg patch applied to the thigh.

### Cohort 7

Cohort 7 evaluated 4 wear times for the 300 μg patch of abaloparatide-TD Formulation W-1 each applied as a single patch administration to the thigh or abdomen using Applicator 3. There were 6 treatment periods for subjects in Cohort 7. Subjects were randomized to 1 of 4 treatment sequences in Treatment Period 1 to 4. Subjects in the randomization treatment stage were treated with 300 μg of abaloparatide-TD with different wear times to the thigh (5, 15, 30 minutes, or 24 hours). Subsequently, all subjects entered the sequential treatment stage starting in Treatment Period 5 and were treated with 300 μg of abaloparatide-TD in the periumbilical region of the abdomen for 15 minutes wear time. In the sixth and final treatment period, subjects were injected with 80 μg of abaloparatide-SC in the periumbilical region of the abdomen. Each treatment period was separated by a washout period of at least 3 days. The design is summarized in Table 7: Treatments for Cohort 7.

TABLE 7
Treatments for Cohort 7
All TD treatments used Formulation W-1, Applicator 2

| Se- | | | | | Period | Period |
|---|---|---|---|---|---|---|
| quence | Period 1 | Period 2 | Period 3 | Period 4 | 5B | 6 |
| 1 | 300 μg, | 300 μg, | 300 μg, | 300 μg, | 300 μg, | SC |
| | Thigh, | Thigh, | Thigh, | Thigh, | Ab- | 80 μg |
| | 5 min | 15 min | 30 min | 24 Hours | domen, | |
| 2 | 300 μg, | 300 μg, | 300 μg, | 300 μg, | 15 min | |
| | Thigh, | Thigh, | Thigh, | Thigh, | | |
| | 15 min | 24 Hours | 5 min | 30 min | | |
| 3 | 300 μg, | 300 μg, | 300 μg, | 300 μg, | | |
| | Thigh, | Thigh, | Thigh, | Thigh, | | |
| | 30 min | 5 min | 24 Hours | 15 min | | |
| 4 | 300 μg, | 300 μg, | 300 μg, | 300 μg, | | |
| | Thigh, | Thigh, | Thigh, | Thigh, | | |
| | 24 Hours | 30 min | 15 min | 5 min | | |

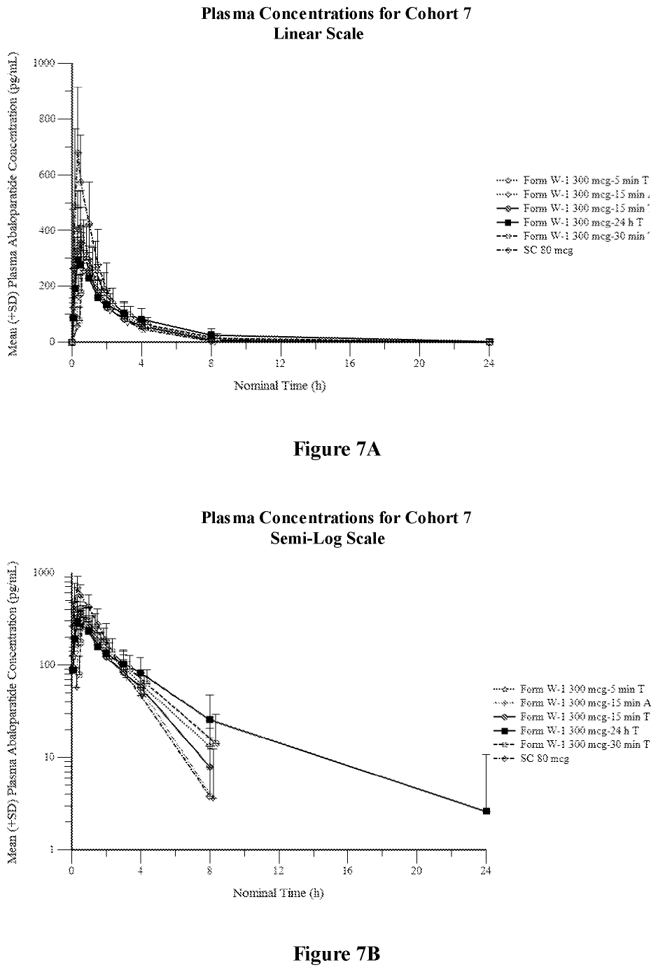

As shown in FIGS. 7A, 7B and 7C, abaloparatide was absorbed slowly, with mean tmax ranging from 27 to 35 minutes for TD treatments, compared to 24 minutes for SCₘₐₓ Similar to assessments in previous cohorts, Formulation W-1 300 μg application to the thigh produced 10% to 25% higher abaloparatide Cₘₐₓ and AUC than application to the abdomen.

For Formulation W-1 300 μg applied to the thigh, AUC_{5 min}=AUC_{24 h}>AUC_{30 min}>AUC_{15 min} (relative bioavailability compared to within-cohort SC was 83%-93%, 81-96%, 73%-77%, and 70%-82%, respectively).

The relative bioavailability for Formulation W-1 compared to SC was similar to that for Formulation W (73-77% vs. 73%, respectively, Thigh, 15 minutes).

Formulation W-1 300 μg applied 5 minutes and 24 hours to the thigh were the most similar to 80 μg abaloparatide-SC with AUC₀₋ₜ and AUC_{0-inf} values achieving 81.1 to 95.8% of those of abaloparatide-SC, respectively. Additionally, compared to the SC 80 μg data pooled across 4 studies, Formulation W-1 300 μg applied for 5 minutes to the thigh provided 12.2% lower to 1% higher AUC and 31.0% lower Cₘₐₓ). The Formulation W-1 300 μg 5 minutes and 24 hours wear time Cₘₐₓ were lower achieving 62.2 and 45.0% of abaloparatide-SC Cₘₐₓ, respectively, for the within-cohort comparisons.

For the Formulation W-1 300 μg patch, the highest systemic exposure levels were reached when the patch was applied for the shortest tested wear time with AUC0-t, AUC0-inf and Cmax values 15 to 21% higher after 5 minutes than after 15 minutes. Increasing the wear time to 30 minutes did not seem to have any impact on systemic exposure, with similar AUC₀₋ₜ, AUC_{0-inf} and Cₘₐₓ values (geometric mean ratios 90 to 106%) after 15 and 30 minutes. As for increasing the wear time to 24 hours, AUC0-inf was increased by 28%, while AUC₀₋ₜ and the Cₘₐₓ were more or less similar with geometric mean ratios of 117 and 88.4%, respectively.

For the Formulation W 400 μg patch (Cohort 6) and for Formulation W-1 300 μg (Cohort 7), higher abaloparatide systemic exposure levels were reached when the patch was applied 5 minutes compared to 15 and 30 minutes. However, when the Formulation W-1 patch was applied for 24 hours, total released dose, and therefore total systemic exposure was increased compared to 15 minutes.

### Model Prediction for BMD

Table 8: Model Prediction for the % Change in BMD in Typical Subject, depicts the model prediction for the percent change in BMD in a typical subject. This assumes a baseline T-score of −2.7, and draws upon dose response studies and population PK/PD modeling. Without wishing to be bound to any particular theory, it is believed that AUC is the key driver of BMD increases.

TABLE 8
Model Prediction for the % Change in BMD in Typical Subject

| | | % Change in | % Change | % Change in | % Change in |
|---|---|---|---|---|---|
| | AUC | BMD | in BMD | BMD | BMD |
| Treatment | (pg · h/mL) | 3 Months | 6 Months | 9 Months | 12 Months |
| Placebo | 0 | 0.29 | 0.42 | 0.52 | 0.60 |
| SC 80 μg | 1018 | 4.39 | 6.28 | 7.74 | 8.98 |
| Formulation | 942 | 4.33 | 6.20 | 7.64 | 8.86 |
| W-1 300 μg | | | | | |
| (Thigh, 5 min) | | | | | |

Overall, abaloparatide application to the thigh consistently provided greater abaloparatide AUC than application to the abdomen, although the difference was less dramatic for Formulation W-1. And the wear-time of 5 minutes provided greater abaloparatide AUC than the wear times for 15 minutes and 30 minutes for both Formulation W 400 μg and Formulation W-1 300 μg. Formulation W-1 300 μg applied to the thigh for 5 minutes provided 83-93% relative bioavailability compared to SC 80 μg, with only slightly lower expected BMD response.

1. A method for treating osteoporosis in a subject in need thereof comprising:
administering daily a trans dermal patch applied to the subject's thigh for about 5 minutes, the patch comprising: 300 μg of abaloparatide, and ZnCl₂, at a molar ratio of 2.2:1 of ZnCl₂:abaloparatide,
wherein the subject is treated for osteoporosis.

2. A method of increasing bone mass density (BMD) in a subject in need thereof comprising:
administering daily a trans dermal patch applied to the subject's thigh for about 5 minutes, the patch comprising: 300 μg of abaloparatide, and ZnCl₂, at a molar ratio of 2.2:1 of ZnCl₂:abaloparatide,
wherein the subject achieves at least a 5% increase in BMD by 6 months.

3. A method for transdermally delivering abaloparatide to a subject in an amount bioequivalent to subcutaneous injection of 80 mcg abaloparatide, the method comprising:
administering daily a trans dermal patch applied to the subject's thigh for about 5 minutes, the patch comprising: 300 μg of abaloparatide, and ZnCl₂, at a molar ratio of 2.2:1 of ZnCl₂:abaloparatide,
wherein an amount of abaloparatide is transdermally delivered to the subject that is bioequivalent to subcutaneous injection of 80 mcg abaloparatide.

4. A once-daily trans dermal system for the delivery of abaloparatide comprising:
a plurality of single-use trans dermal patches, each loaded with about 300 μg of abaloparatide, and ZnCl₂ at a molar ratio of 2.2:1 of ZnCl₂:abaloparatide; and
instructions to administer one of said trans dermal patches once daily to the thigh for about 5 minutes.

5. An aqueous formulation suitable for coating a trans dermal patch wherein the aqueous formulation comprises 300 μg of abaloparatide, zinc at a molar ratio of 2.2:1 of Zn:abaloparatide.

6. The method, system or formulation according to any of the preceding claims, wherein the patch or formulation further comprises hydrochloric acid.

7. The method, system or formulation according to claim 6, wherein the mole ratio of hydrochloric acid to zinc chloride is at least 0.025.

8. The method, system or formulation according to any of the preceding claims, wherein the pH of the patch or formulation is about 4.5.

9. The method, system or formulation according to any of the preceding claims, wherein the pH of the patch or formulation is between about 4 and 4.75.


SEQUENCE LISTING (1 sequence)

SEQ ID NO: 1 - protein, 34 aa, Artificial Sequence
  synthetic peptide
  MOD_RES 29: Aib
  AMIDATION 34
AVSEHQLLHDKGKSIQDLRRRELLEKLLXKLHTA